SEQUENCE LISTING (8 sequences)

SEQ ID NO: 1 - protein, 179 aa, Citrobacter rodentium
  ref <308>: DATABASE ACCESSION NUMBER: AF311901
  ref <309>: DATABASE ENTRY DATE: 2001-09-24
  ref <313>: RELEVANT RESIDUES IN SEQ ID NO: (1)..(179)
MDYIGRCIMSSSLSGITFTTFLTNNHASWNRLTRNLYISNTDIVDEVQLKARTQQRHHRIFPEIDDCSIIHFRGKEYFVRFIKDDKSDDYIYKVQKMTQESGCFSKIFGFLYGGVTKTLECKLNERHITPLSSTWYPRSSLEGFLTERGLSSLLRRVQSTENVPLSESATLNPLSGMMV

SEQ ID NO: 2 - protein, 176 aa, Escherichia coli
  ref <308>: DATABASE ACCESSION NUMBER: AF022236
  ref <309>: DATABASE ENTRY DATE: 1998-04-25
  ref <313>: RELEVANT RESIDUES IN SEQ ID NO: (1)..(176)
MRYIGRCMMSSSLSGITFTTSLTSHASWNKLTRTLHISNDDIVDELQLKARMQQRHHRVFAGIDDYSIINFRGKDYAVRFIKDKDNYVYKVNKITQESGCFSRIFSFLYGGVTKALESKLNERHITPLSSTWFPRTALEGFLTERGLSSLLRRVQSTEGVNPPENTTLNSLSGVTV

SEQ ID NO: 3 - DNA, 23 bp, Artificial
  PCR primer for espH-5 (forward)
aactgcagaagaggagcactcgt

SEQ ID NO: 4 - DNA, 23 bp, Artificial
  PCR primer for espH-5 (reverse)
gcgtcgaccatgatacatctccc

SEQ ID NO: 5 - DNA, 23 bp, Artificial
  PCR primer for espH-3 (forward)
gcgtcgaccctttgtcaggcatg

SEQ ID NO: 6 - DNA, 23 bp, Artificial
  PCR primer for espH-3 (reverse)
gctctagaaatctgctcctgccg

SEQ ID NO: 7 - DNA, 25 bp, Artificial
  PCR primer for IL-12p40 (forward)
cagaagctaaccatctcctggtttg

SEQ ID NO: 8 - DNA, 24 bp, Artificial
  PCR primer for IL-12p40 (reverse)
ccggagtaatttggtgctccacac

(19) United States
(12) Patent Application Publication
(10) Pub. No.: US 2008/0317875 A1
(43) Pub. Date: Dec. 25, 2008

(54) Inhibitor of PI3 kinase-dependent inflammatory cytokine synthesis and method for inhibiting the same

(76) Inventors: Shigeo Koyasu, Tokyo (JP); Masashi Ohtani, Tokyo (JP); Chihiro Sasakaw, Tokyo (JP); Sei Yoshida, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/822,664

(22) Filed: Jul. 9, 2007

(30) Foreign Application Priority Data
Jul. 10, 2006 (JP) 2006-189417

(51) Int. Cl. A61K 33/00; A61P 37/00; A61P 9/10; A61P 31/04

(52) U.S. Cl. 424/722

(57) ABSTRACT

The present invention provides a novel inhibitor for inhibiting synthesis of a PI3 kinase-dependent inflammatory cytokine in vivo in a vertebrate and a method for inhibiting the same. More particularly, the present invention provides a suppressor for suppressing a cell-mediated immune response and a method for suppressing the same, as well as an activator for activating a humoral immune response and a method for activating the same. In the present invention, by administering Li ion to a living body to inhibit synthesis of an inflammatory cytokine, a cell-mediated immune responses can be suppressed, and immune-mediated inflammatory disorders (IMIDs) can be treated.

### CROSS REFERENCE TO RELATED APPLICATIONS

This application claims the benefit of priority to Japanese Patent Application No. 2006-189417, filed on Jul. 10, 2006, which is incorporated herein by reference.

### BACKGROUND OF THE INVENTION

1. Field of the Invention

The present invention relates to inhibitors which inhibit synthesis of a PI3 kinase-dependent inflammatory cytokine in vivo in a vertebrate and methods for inhibiting the same.

2. Description of the Related Art

Phosphoinositide-3 (PI3) kinases, a subfamily of lipid kinases, have the activity to phosphorylate phosphatidylinositol, one of the intracellular signaling molecules, and regulate many cellular functions by phosphorylation. Meanwhile, immune responses, which are the defense mechanism against foreign substances in animals, consist of two mechanisms: cell-mediated immune responses and humoral immune responses. It has been considered that, among CD4-positive T cells, activation of Th1 cells induces cell-mediated immune responses, whereas activation of Th2 cells induces humoral immune responses. In both of these mechanisms, dendritic cells play a pivotal role.

The dendritic cells express and secrete cytokines such as interleukin 12 (IL-12) upon stimulation. Cytokines are physiologically active substances regulating the functions of various types of cells including immune cells. The cytokines include not only interleukins but also interferons, chemokines, etc. Among these, IL-12, an inflammatory cytokine, plays a role in triggering cell-mediated immune responses by acting on T cells.

It has recently been elucidated that the expression of this IL-12 in dendritic cells is negatively regulated by PI3 kinase activity (see Fukao, T., Tanabe, M., Terauchi, Y., Ota, T., Matsuda, S., Asano, T., Kadowaki, T., Takeuchi, T. and Koyasu, S. (2002) “PI3K-mediated negative feedback regulation of IL-12 production in dendritic cells.” Nat. Immunol. 3: 875-881). Further, GSK3β, which is one of the proteins serving as the substrates for Akt, a kinase downstream of PI3 kinases, is inactivated in its own kinase activity when phosphorylated. It has recently been reported that GSK3β positively regulates IL-12 expression in monocytes (see M. Martin, K. Rahani, R. S. Jope, S. M. Michalek, Nat. Immunol. 6, 777 (2005)). It is therefore concluded that activation of a PI3 kinase results in suppression of the IL-12 expression through the phosphorylation of GSK3.

Under such circumstances, it has come to be considered that, by regulating PI3 kinase-dependent inflammatory cytokines, such as IL-12, in vivo in vertebrates, the balance between the cell-mediated immune responses and the humoral immune responses can be regulated, and, further, by regulating this balance, diseases in which immunity system is involved can be treated.

Thus, the object of the present invention is to provide novel inhibitors which inhibit synthesis of a PI3 kinase-dependent inflammatory cytokine in vivo in a vertebrate and methods for inhibiting the same.

### SUMMARY OF THE INVENTION

When bacteria are infected into host cells, in many cases, intracellular PI3 kinases are activated for a long term. However, enteropathogenic Escherichia coli (EPEC), a type of pathogenic E. coli causing disorders such as diarrhea and enteritis, when cultured with established dendritic cells, promptly inactivates PI3 kinase activity in the dendritic cells. Here, an EspH-defective mutant EPEC strain was constructed by introducing a mutation into the espH locus in EPEC. This espH mutant or the wild-type strain was then co-cultured with established dendritic cells. As a result, it was found that PI3 kinase activity is suppressed in dendritic cells infected with the wild-type, whereas the inactivation of PI3 kinase did not occur in cells infected with the espH mutant (Example 1). Next, a similar co-culture experiment was performed using a primary culture of dendritic cells isolated from murine bone marrow-derived dendritic cells (BMDCs). The wild-type caused a decrease in PI3 kinase activity in BMDCs and sustained high expression of IL-12, whereas the espH mutants caused a decrease in suppression of phosphorylation and also lowered the increase in IL-12 expression (Example 2). The findings showed that the EspH protein increases IL-12 expression by suppressing the PI3 kinase activity in dendritic cells.

Next, the wild-type of Citrobacter rodentium, a pathogenic E. coli infectious to mice, or its espH mutant, was orally administered to mice and infection to the large intestine was examined. The wild-type bacteria induced inflammation abd their proliferation and colonization was observed, whereas the espH mutant resulted in a low infection (Example 3). Further, knockout mice lacking the PI3 kinase gene were infected with wild-type Citrobacter rodentium or its espH mutant. As a result, the difference in infection depending on the presence or absence of the espH mutation was not observed in the knock-out mice unlike the wild-type mice (Example 4). In addition, in a similar infection experiment using mice transplanted with knockout mouse-derived bone marrow (which contains dendritic cells), the same phenotype as that exhibited when knockout mice were infected was observed (Example 5). Meanwhile, an experiment to induce differentiation of knockout mouse-derived T cells revealed that chemokine secretion by T cells is regulated by PI3 kinase (Example 6). These findings indicated that the EspH protein has the effect of inducing cell-mediated immune responses of host animals via increased IL-12 expression in dendritic cells by suppression of PI3 kinase.

Further, when GSK3β activity was inhibited by adding LiCl or SB216763 in isolated dendritic cells, IL-12 expression was found to decrease (Example 7). Then, mice were infected with C. rodentium and 3 days later LiCO₃ was added to their drinking water. As a result, the symptom of bacterial infection was relieved and, at the same time, the expression of interferon γ (IFN-γ) was suppressed (Example 8). Since IFN-γ expression is induced by IL-12 in Th1 cells which have the function of enhancing cell-mediated immune responses, the results of these experiments revealed that, not only in the cultured cells but also in vivo, expression of IL-12 was suppressed by administration of Li ions. Further, since IFN-γ plays a pivotal role in Th1 cell differentiation and proliferation, Li ions were shown to suppress production of inflammatory cytokines as well as murine cell-mediated immune response and to activate humoral immune responses. It was therefore shown that the administration of Li ions to a living body can suppress cell-mediated immune response in vivo and infection by bacteria. Based on the above-described new findings, the inventors have accomplished the following invention.

Namely, in one embodiment of the present invention, an inhibitor for inhibiting in vivo synthesis of a PI3 kinase-dependent inflammatory cytokine in a vertebrate contains Li ion as an active ingredient. The cytokine may be IL-12. The inhibitor may be administered by injection or oral administration.

In another embodiment, a suppressor of cell-mediated immune responses in a vertebrate contains Li ion as an active ingredient. The suppressor may be administered by injection or oral administration.

In another embodiment, an activator of humoral immune responses in a vertebrate contains Li ion as an active ingredient. The activator may be administered by injection or oral administration.

In yet anther embodiment, a therapeutic agent for a disease resulting from synthesis of a PI3 kinase-dependent inflammatory cytokine contains Li ion as an active ingredient. The cytokine may be IL-12. The therapeutic agent may be administered by injection or oral administration.

In another embodiment, a therapeutic agent for a disease resulting from an increase in the ratio of cell-mediated immune responses to humoral immune responses contains Li ion as an active ingredient. The disease may be a disorder resulting from proliferation of pathogenic E. coli, an immune-mediated inflammatory disorder (IMID), habitual miscarriage, an organ-specific autoimmune disease based on delayed type hypersensitivity, hepatic disorder, or arteriosclerosis. The therapeutic agent may be administered by injection or oral administration.

In yet another embodiment, a suppressor for suppressing in vivo proliferation of pathogenic E. coli in a vertebrate contains Li ion as an active ingredient. The suppressor may be administered by injection or oral administration.

In yet another embodiment, a method for inhibiting synthesis of a PI3 kinase-dependent inflammatory cytokine in a human or a nonhuman vertebrate includes the step of administering Li ion to the vertebrate. The cytokine may be IL-12. The Li ion may be administered by injection or oral administration.

In yet another embodiment, a method for suppressing cell-mediated immune responses in a human or a nonhuman vertebrate includes the step of administering Li ion to the vertebrate. The Li ion may be administered by injection or oral administration.

In yet another embodiment, a method for activating a humoral immune response in a human or a nonhuman vertebrate includes the step of administering Li ion to the vertebrate. The Li ion may be administered by injection or oral administration.

### BRIEF DESCRIPTION OF THE DRAWINGS

FIG. 1 shows results from an experiment to co-culture EPEC or its mutant and established dendritic cells in Example 1 of the present invention. Each of the amounts of phosphorylated Akt protein (upper panels in a, b, and c) and α-tubulin (lower panels in a and b) as the internal control is shown as intensities of bands detected by Western blotting in cells after a lapse of designated hours (denoted as H) from infection with the bacterial strains shown on top of a, b, and c.

FIG. 2 shows results from an experiment to co-culture EPEC or its mutant and bone marrow-derived dendritic cells (BMDCs) in Example 2 of the present invention. BMDCs harvested from the mouse strain shown at the top left corner were infected with the bacterial strains shown on the left end (the wild-type in b) and cultured. Panels a and b show images obtained by microscopic observation after visualization of actin (left column) or DNA (middle column) along with their superimposed image (right column). Bacteria are displayed as small spots due to DNA visualization. Panel c shows, like FIG. 1, changes in the amount of phosphorylated Akt in BMDCs after infection with the wild-type or the mutant. Panel d shows the result of relative measurement of cytokine expression (the level of IL-12p40 mRNA) in the cells after a lapse of designated hours (denoted as H) from the infection. The black, diagonally-striped, and white vertical bars indicate infection by the wild-type, TTSS mutant, and espH mutant, respectively.

FIG. 3 shows results from an experiment to infect B10.D2 mice with C. rodentium or its mutant in Example 3 of the present invention. Panel a shows the degree of infection in the colon after a lapse of designated number of days from an oral infection. The black and white vertical bars indicate the number of bacteria (cfu) of the wild-type or the mutant, respectively. Panel b shows changes in the tissue weight of the colon excised after a lapse of designated number of days from the infection, and white and black circles indicate wild-type C. rodentium and its espH mutant, respectively. Panels in c show results from microscopic (upper) and histopathological (lower) observations of the colon excised on day 12 after the infection with the wild-type (left) or the mutant (right). In each histopathological image, the right panel shows an enlarged view of the boxed area in the left panel. Panel d shows the production of IFN-γ in the mesenteric lymph node of the uninfected mice or the mice infected with the wild-type or the mutant.

FIG. 4 shows results from an experiment to infect PI3 kinase knockout (KO) BALB/c mice or wild-type BALB/c mice with C. rodentium or its mutant in Example 4 of the present invention. Panels a and b show the number of infecting bacteria (a) and the tissue weight (b) of the colon of the mice infected with wild-type C. rodentium (black vertical bar) or its espH mutant (white vertical bar). Panels c and d show the results from the observation of the colon excised from wild-type (left) or knockout (right) mice infected with the wild-type C. rodentium (c) or the mutant (d). Like FIG. 3c, a macroscopic view (upper), a histopathological view (lower left), and its enlarged view (lower right) are shown for each macroscopic image.

FIG. 5 shows results from an experiment to infect bone marrow chimeric mice with C. rodentium in Example 5 of the present invention. Panels a, b, and c show the temporal changes in the number of bacteria (a) and tissue weight (b) as well as the result from the observation (c) of the colon excised on day 12 after the infection of the chimeric mice with the wild-type C. rodentium. The results in transplantation experiments of the bone marrows of p85α knockout mice to p85α heterozygous mice are shown in white vertical bars in a and b, and in the left half in c, while the results when the bone marrows of heterozygous mice were transplanted to heterozygous mice are shown in black vertical bars in a and b, and in the right half in c. In c, like in FIG. 3c, a macroscopic view (upper), a histopathological view (lower left), and its enlarged view (lower right) are shown for each colon sample.

FIG. 6 shows results from an experiment to induce differentiation of T cells derived from knockout or heterozygous mice at PI3 kinase locus in Example 6 of the present invention. T cells derived from the spleen of p85α knockout mice (PI3KKO) or p85α heterozygous mice (PI3KHT) were cultured without the addition of any drug (−), with simultaneous addition of two types of antibody (α-CD3 and α-CD28), or two types of differentiation inducers (PMA and ionomycin). The result from the quantification performed for each culture 48 hours later of the chemokine (MIP-2) secreted by cells into the conditioned medium is shown on the vertical axis.

FIG. 7 shows results from an experiment to culture BMDCs to which GSKβ inhibitor was added in Example 7 of the present invention. In A, each of the amounts of phosphorylated Akt protein (upper), and α-tubulin (lower) as the internal control, are shown as intensities of bands detected by Western blotting in cells after a lapse of designated hours (denoted as H) from infection with wild-type EPEC or its espH mutant. B shows the result from the measurements obtained by ELISA of the cytokine expression (the production of the 12pIL-70 protein) in BMDCs activated by LPS stimulation. “NT” indicates measurement without stimulation; “SB” and “LiCl” indicate measurement with the addition SB216763 or LiCl, respectively, prior to stimulation; and “−” indicates measurement without the addition.

FIG. 8 shows results from an experiment to administer Li ions to B10.D2 mice infected with C. rodentium in Example 8 of the present invention. A and B show the results from the macroscopic (upper) and histopathological (lower) observation of the colon of mice in the control group not receiving LiCO₃ (A) or mice in the experimental group receiving LiCO₃ (B); in each histopathological image, the right panel shows an enlarged view of the boxed area in the left panel. C shows changes in the tissue weight of the colon excised after a lapse of designated number of days from infection, and black, gray, and white circles indicate the result obtained from the mice before administration, in the control group, and the experimental group, respectively. D and E show the number of bacteria (cfu) infected in the colon (D), and the production of IFN-γ (pg/ml) in isolated mesenteric lymph node (E), of the mice in the experimental group (+) or the control group (−). Asterisks denote significant difference (p<0.05).

### DETAILED DESCRIPTION OF THE PREFERRED EMBODIMENTS

The present invention is directed to novel PI3 kinase inhibitors and their use. Embodiments of the present invention accomplished based on the above-described findings are hereinafter described in detail by giving Examples. Unless otherwise explained, methods described in standard sets of protocols such as J. Sambrook and E. F. Fritsch & T. Maniatis (Ed.), “Molecular Cloning, a Laboratory Manual (3rd edition), Cold Spring Harbor Press and Cold Spring Harbor, N.Y. (1989); and F. M. Ausubel, R. Brent, R. E. Kingston, D. D. Moore, J. G. Seidman, J. A. Smith, and K. Struhl (Ed.), “Current Protocols in Molecular Biology,” John Wiley & Sons Ltd., or alternatively, their modified/changed methods are used. When using commercial reagent kits and measuring apparatus, unless otherwise explained, protocols attached to them are used. The object, characteristics, and advantages of the present invention as well as the idea thereof are apparent to those skilled in the art from the descriptions given herein. It is to be understood that the embodiments and specific examples of the invention described herein below are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

EspH Protein Derived from Pathogenic E. coli

Many gram-negative pathogenic bacteria, including EPEC, introduce various effector molecules into cells in a host animal through the type III secretion system (TTSS), thereby affecting characteristics of the cells. Enteroadherent bacteria, such as EPEC and enterohemorrhagic Escherichia coli (EHEC), adhere to epithelial cells in the intestinal tract and destroy their microvilli (formation of attaching and effacing (A/E) lesions). The A/E lesions are caused by effector molecules secreted through the TTSS; many of genes encoding these effector molecules belong to a pathogenic gene family referred to as locus of enterocyte effacement (LEE). The EspH protein, an active ingredient according to the present invention, has been previously known as one of the LEE gene products (see Xuanlin Tu, Israel Nisan, Chen Yona, et al. (2003) “EspH, a new cytoskeleton-modulating effector of enterohaemorrhagic and enteropathogenic Escherichia coli.” Molecular Microbiology, 47(3), 595-606).

LEE is present in EHEC and EPEC, as well as other pathogenic E. coli which have nearly the same A/E pathogen as EPEC and are infectious to other animals, exemplified by Citrobacter rodentium which is infectious to mice (see Wanyin Deng, Yuling Li, Bruce A. Vallance, and B. Brett Finlay (2001) “Locus of Enterocyte Effacement from Citrobacter rodentium; and Sequence Analysis and Evidence for Horizontal Transfer among Attaching and Effacing Pathogens.” Infection and Immunity, 69(10), 6323-6335). By infecting Citrobacter rodentium (which can be regarded as a mouse EPEC equivalent) and its mutants into mice, model experiments for analyzing A/E lesions can be conducted.

Therefore, the EspH proteins that can be used for the present invention include a protein derived from Citrobacter rodentium, one of the pathogenic E. coli strains, and having the amino acid sequence shown in SEQ ID NO: 1, as well as a protein derived from EPEC and having the amino acid sequence shown in SEQ ID NO: 2, as described in the Examples. Further, any protein derived from a pathogenic E. coli strain having a highly conserved LEE and causing a similar A/E lesion, such as EHEC, can also be used.

### Inhibition of PI3 Kinase by EspH Protein

As shown in Examples 1 and 2, since the EspH protein has an inhibitory activity on PI3 kinases, the EspH protein can be used to inhibit a PI3 kinase. The inhibition of a PI3 kinase may be performed by using any form of the EspH protein as long as the inhibitory activity of the EspH protein on the PI3 kinase is utilized. For example, not only the whole EspH protein but also a protein composing of a part of the EspH protein may be used, as long as the active center of the EspH protein inferred to be the minimal requirement is included. The reaction system to utilize this inhibitory activity of the EspH protein may be an in vitro system or an in vivo system; for example, an in vitro reconstitution system, as well as an in vivo reconstitution system, such as one within a cell extract, a tissue culture, or an organism, may be used as the reaction system. The inhibitory effect may be brought in either way where the EspH protein directly inhibits a PI3 kinase, or the EspH protein indirectly inhibits a PI3 kinase by regulating the function of another upstream molecule transmitting signals to the PI3 kinase, thereby utilizing the function of this regulated molecule.

Thus, the EspH protein is useful as an inhibitor of PI3 kinase. The PI3 kinase inhibitor may take any dosage form, and any kind of additive may be included as long as it does not inhibit the inhibitory activity on PI3 kinase by the EspH protein.

The methods for administering the EspH protein to cells include: incorporating a gene encoding the EspH protein into an expression vector such as a plasmid or a viral vector and transducing the gene into cells; preparing a fusion protein by fusing the EspH protein to the cell-membrane transduction domain of Tat protein (Tyr-Gly-Arg-Lys-Lys-Arg-Arg-Gln-Arg-Arg-Arg) of HIV-1; and infecting with bacteria, such as EPEC, which allow introduction of the EspH protein during the infection.

Cells into which the EspH protein is to be introduced are not limited as long as they contain PI3 kinase, and the EspH protein acts effectively as a PI3 kinase inhibitor on the target cells. Particularly suitable are dendritic cells. This is because, since the expression of an inflammatory cytokine in dendritic cells can be increased, immune responses within an individual can be regulated, and/or therapeutic agents for diseases such as allergy can be provided, by allowing dendritic cells in an individual to contain the EspH protein, as will be further described later.

### Promotion of Synthesis of the Inflammatory Cytokine Secretion by the EspH Protein

Suppression of PI3 kinase can promote synthesis of PI3 kinase-dependent inflammatory cytokines. In this case, the previously-mentioned dendritic cell is a suitable example of the cell into which the EspH protein is introduced. Other examples include cells in which inflammatory cytokine secretion is regulated in a PI3 kinase-dependent manner, such as monocytes and macrophages (see, e.g., Guha, M. and Mackman, N. (2002) “The phosphatidylinositol 3-kinase-Akt pathway limits lipopolysaccharide activation of signaling pathways and expression of inflammatory mediators in human monocytic cells.” J. Biol. Chem. 277: 32124-32132), Salmonella-infected epithelial cells (see e.g., Huang, F. C., Li, Q. and Cherayil, B. J. (2005) “A phosphatidyl-inositol3-kinase-dependent anti-inflammatory pathway activated by Salmonella in epithelial cells.” FEMS Microbiol. Lett. 243: 265-270), and T cells (refer to Example 6).

The inflammatory cytokine whose synthesis is promoted is mainly IL-12 in the case of dendritic cells, but synthesis of other inflammatory cytokines such as chemokines secreted from T cells can be also promoted (see Example 6). In addition, syntheses of tumor necrosis factor (TNF) secreted from the above-mentioned macrophages, tissue factor (TF), chemokines and cytokines secreted from epithelial cells stimulated by Salmonella infection etc. through the Toll-like receptor, can be promoted by inhibiting PI3 kinase.

Thus, the EspH protein is useful as a promoter for promoting inflammatory cytokine synthesis. In this case, the promoter which promotes inflammatory cytokine synthesis may take any dosage form; any kind of additive may be included as long as it does not inhibit the inhibitory activity on PI3 kinase by the EspH protein.

Further, it is possible to promote cytokine synthesis in a cell in which inflammatory cytokine synthesis is regulated by IL-12, as a result of the promotion of the IL-12 synthesis in dendritic cells. For example, in the presence of dendritic cells to which the EspH protein has been administered, IL-12 secretion from dendritic cells can be increased, resulting in promotion of synthesis of T-cell-secreted cytokines, such as IFN-γ, in T cells.

Thus, the EspH protein is also useful as a promoter for promoting cytokine synthesis in T cells in the presence of dendritic cells to which the EspH protein has been administered. Here, the promoter for promoting cytokine synthesis in T cells may take any dosage form; any kind of additive may be included as long as it does not inhibit the inhibitory activity on PI3 kinase by the EspH protein.

It should be noted that if occurrence of an increase in IL-12 expression is not desirable when a PI3 kinase is inhibited by the EspH protein in dendritic cells, for example, the PI3 kinase can be inhibited while avoiding the increase in IL-12 expression by the use of IL-12-deficient dendritic cells or by the administration of an IL-12 inhibitor to dendritic cells.

### Regulation of Immune Responses by the EspH Protein

Inflammatory cytokines, IL-12 in particular, have a strong ability to activate cell-mediated immune responses, as shown in Examples 3 and 4. This indicates that the EspH protein is also useful as an activator of the cell-mediated immune responses and capable of enhancing the cell-mediated immune responses through an increase in inflammatory cytokine expression by being administered to a vertebrate. Besides, since a balance is maintained between the two immune response mechanisms, i.e. the cell-mediated immune responses and the humoral immune responses in vertebrates, the EspH protein can also be used as a suppressor of the humoral immune responses.

In the above case, the dose and dosage form of the EspH protein may be appropriately selected so that the suppressor will be the most effective for the purpose of regulating immune responses. For example, the EspH protein itself may be administered to an individual; alternatively, the gene encoding the EspH protein may be administered to an individual. The mode of administration of the EspH protein to an individual includes, but not particularly limited to, application, spraying, injection, and infection of an infectant. Likewise, the mode of administration of the gene may be any method generally used for introducing and expressing a gene in vivo, such as administration of an expression vector.

In summary, the present invention can provide regulators capable of regulating animal immune responses and methods for such regulation by using the EspH protein.

### Use of the EspH Protein in Treatment of Diseases

An allergic reaction is a disease caused by an overreaction of the humoral immune system to an allergen. Accordingly, the allergic disease can be treated by administering the EspH protein according to the present invention, thereby suppressing the humoral immune responses.

For example, in the case of skin allergy, by using a dosage form which can deliver the EspH protein to subcutaneous dendritic cells causing allergy, e.g. an ointment, the present invention can provide an antiallergic agent for skin allergy. Alternatively, depending on the form of an allergic reaction, other general modes of administrations and dosage forms can be used. It should be noted that if occurrence of symptoms of inflammation or other conditions accompanying increased cell-mediated immune responses is not desirable when suppressing the humoral immune responses as described above, the problem can be avoided by administering also a symptomatic antiinflammatory agent which is effective on a reaction occurring downstream of reactions in the dendritic cells.

The present invention further provides a therapeutic agent for cancer treatment. For example, in cancer immunotherapy, dendritic cells being allowed to present a cancer antigen are administered to a cancer patient. In this case, by introducing the EspH protein into the dendritic cells to be administered to a patient, the IL-12 expression can be increased, thereby making it possible to enhance the production of cancer-specific cytotoxic T cells after the dendritic cells are transplanted.

### Suppression of Inflammatory Cytokine Expression by Li Ions and its Use

In the PI3 kinase signal transduction pathway in cells of a vertebrate (either a human or a nonhuman vertebrate), Li ions act downstream of the PI3 kinase to suppress expression of inflammatory cytokines such as IL-12 (see Example 7). Li ions are therefore effective in treating diseases resulting from expression of PI3 kinase-dependent inflammatory cytokines.

Since inflammatory cytokines, such as IL-12, have a strong ability to activate cell-mediated immune responses, Li ions are inferred to be effective as a suppressor of cell-mediated immune responses and an activator of humoral immune responses. In fact, in vivo in vertebrates, cytokines have the function of suppressing cell-mediated immune responses and activating humoral immune responses, thereby suppressing proliferation of pathogenic bacteria (see Example 8).

Diseases for which PI3 kinase-dependent inflammatory cytokines and cell-mediated immune responses are responsible and in which the ratio of cell-mediated immune responses to humoral immune responses is high include: inflammatory bowel diseases (chronic ulcerative colitis and Crohn's disease), rheumatic arthritis, immune-mediated inflammatory disorders such as multiple sclerosis (see Vizcarra C., J Infus Nurs. vol. 26, pp 319-25, 2003; and Williams J P and Meyers J A., Am J Manag Care. suppl, pp. S664-81, 2002), organ-specific autoimmune diseases based on delayed type hypersensitivity, hepatic disorder, and arteriosclerosis. Li ions are useful as a therapeutic agent for all these diseases. In addition, since the a ratio of the cell-mediated immune responses to the humoral immune responses is considered to be the cause of miscarriage in patients with habitual miscarriages, Li ions can potentially act effectively on suppression of a miscarriage.

The form and dosage forms of Li ions are not limited, but it is preferred to be administered in a form of lithium salt solution, such as lithium chloride solution, lithium sulfate solution, lithium hydroxide solution, and lithium carbonate solution.

The mode of administration of Li ions to a vertebrate is not particularly limited; for example, Li ions may be orally administered by being contained in drinking water, diets, tablets, etc. Alternatively, Li ions may be administered locally by any method which can deliver Li ions locally to the site where suppression of bacterial infection or activation of humoral immune responses is desired, for example, by injecting an injection solution containing Li ions to a site infected with bacteria or its vicinity.

### EXAMPLES

Embodiments of the present invention will be described in more details by way of Examples and Drawings hereinbelow.

### Example 1

### Experiment of Co-Culture of Enteropathogenic E. coli (EPEC) and Dendritic Cells

It has been shown that PI3 kinase activity is inhibited by the EspH protein by examining PI3 kinase activity observed in established myeloid dendritic cell line DC2.4 upon infection with EPEC using the degree of phosphorylation of Akt protein, a substrate of phosphorylation reaction by the PI3 kinase, as an indicator.

### Co-Culture.

Established dendritic cell line DC2.4 (provided by Dr. Kenneth Rock of Dana, Farber Cancer Institute) were cultured in RPMI1640 medium (Invitrogen) containing 10% fetal bovine serum (SIGMA) at 37° C. in 5% CO₂. Meanwhile, EPEC strain 2348/69 (provided by Sasakawa Lab., Institute of Medical Science, University of Tokyo) or their TTSS mutant (provided by Sasakawa Lab., Institute of Medical Science, University of Tokyo) and nonpathogenic E. coli strain MC1064 (provided by Sasakawa Lab., Institute of Medical Science, University of Tokyo) were each cultured overnight in LB medium (10 g/1 l Polypepton (Nihon Pharmaceutical Co., Ltd., 5 g/1 l Bacto™ Yeast Extract (BD) 5 g/1 l NaCl (Wako), 1.2 ml/1 l 4 N-NaOH (Wako)), and then diluted at 1:10 with DMEM medium (SIGMA), followed by 2 hr incubation at 37° C. To the cultured cells transferred to 6-well culture plates were added the culture of each of the above-mentioned bacterial strains at a multiplicity of infection (MOI) of 100, followed by 3 hr incubation at 37° C. in RPMI1640 medium containing 10% fetal bovine serum.

### Western Blotting.

Part of the cultured cells after a lapse of designated time from bacterial infection was washed in phosphate buffer solution (PBS). For each strain, cells were solubilized with SDS sample solubilization buffer and boiled for 5 min. These samples were subjected to SDS-polyacrylamide gel electrophoresis and then transferred to a PVDF transfer membrane (BIORAD). The transfer membrane was blocked in Tris-buffered saline (TBS) containing 5% skim milk and 0.05% Tween-20 and then washed 3 times (10 min each) in TBS containing 0.05% Tween-20. As the primary antibody, anti-phosphorylated Akt antibody (Cell Signaling) was diluted in TBS containing 5% BSA at 1:500 and then bound to the aforementioned transfer membrane overnight at 4° C. As a control, anti-α-tubulin antibody (SIGMA) was diluted in TBS at 1:1000, and then bound to the transfer membrane in the same manner at room temperature. Then, the transfer membranes were treated with the secondary antibody (SIGMA) diluted in TBS at 1:2000 for 1 hr at 37° C., and the phosphorylated Akt protein on the transfer membrane was detected using an ECL Western Blotting Detection Reagent (Amersham Biosciences).

Construction of an espH Mutant and Co-Culture Experiment.

First, an EPEC mutant in which the function of the EspH protein was reduced was prepared. As the mutant, while either a hypomorph or an amorph may be used, an amorph with a defect in the espH was constructed in this case. (For details, see refer to Matsuzawa, T., Kuwae, A., Yoshida, S., Sasakawa, C. and Abe, A. (2004) “Enteropathogenic Escherichia coli activates the RhoA signaling pathway via the stimulation of GEF-H1.” EMBO J. 23:3570-3582.) Using the resulting espH mutant, a co-culture experiment was performed with DC 2.4 cells in the same manner as described above for the wild-type. Western blotting was performed for detection of phosphorylated Akt protein using the cells infected with the espH in the above-mentioned manner. It should be noted that in this experiment, since bacterial infection involves dendritic cell stimulation, dendritic cells were not particularly stimulated. However, dendritic cells may be stimulated by administrating a Toll-like receptor ligand, such as lypopolysaccharide, CpG-DNA, etc.

### Results.

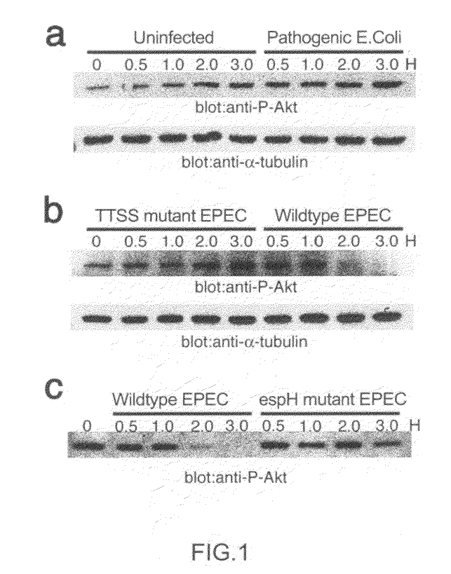

Infection of nonpathogenic E. coli into dendritic cells resulted in a gradual increase in the phosphorylation of Akt protein (FIG. 1a). In contrast, infection of wild-type EPEC into dendritic cells resulted in a rapid decrease in the phosphorylation of Akt protein, followed by its practical disappearance within 3 hr (FIG. 1b right). On the other hand, use of the mutant in which the secretory function by TTSS was deleted did not lead to such suppression of phosphorylation (FIG. 1b left). These results suggested that such suppression of phosphorylation is caused by a factor in which TTSS is involved. Thus, the mutant with a defect in the espH gene was used in the same manner. As a result, the decrease in phosphorylation disappeared as had been expected (FIG. 1c right). It should be noted that in a and b, in order to avoid secondary changes caused by phagocytosis in nonpathogenic E. coli and the TTSS mutant of EPEC, cytochalasin D, which is capable of inhibiting phagocytosis, was added. The same results were obtained in the absence of cytochalasin D.

These findings indicated that the suppression of PI3 kinase activity by EPEC in dendritic cells is associated with the action of EspH protein, and thus, the PI3 kinase activity within dendritic cells can be inhibited by introducing the EspH protein into the dendritic cells. It was therefore clarified that the EspH protein is useful as a PI3 kinase inhibitor.

### Example 2

### Experiment of Co-Culture of EPEC and Bone Marrow-Derived Dendritic Cells

Next, IP3 kinase activity and IL-12 expression were examined when EPEC is infected into the primary culture of bone marrow-derived dendritic cells (BMDCs) in the same manner as in Example 1. It has been shown that administration of the EspH protein to primary cultured dendritic cells results in suppression of intracellular IP3 kinases and then synthesis of IL-12, as is the case with established dendritic cells.

### Isolation and Co-Culture of Bone Marrow Cells.

Cells were harvested from the bone marrow of the femur and tibia of B10.D2 mice with a syringe fitted with a needle and transferred to RPMI 1640 complete medium containing 10 ng/ml granulocyte-macrophage colony-stimulating factor (GM-CSF, PeproTech EC) in 6-well culture plates, and culture was started.

The medium was changed 2 and 4 days after the start of culture to remove granulocytes. Six days after the start of culture, loosely adherent cells were recovered by gentle pipetting. Using N418 magnetic beads and a cell sorter (Myltenyi Biotec), cells expressing CD 11 were isolated as BMDCs. Using the isolated BMDCs, co-culture with EPEC was performed like the established dendritic cells in Example 1, and the following analysis was conducted.

### Morphological Observation of Cells and Nuclei.

The infected cells were transferred to coverslips and fixed with PBS containing 4% paraformaldehyde for 20 min at room temperature. For detection of actin, labeling was performed by diluting rhodamine-phalloidin (Molecular Probe) at 1:100 in TBS and incubated with the coverslips at 37° C. for 1 hr. In addition, for detection of DNA, nuclear staining was performed by diluting TO-PRO-3 (Molecular Probe) at 1:100 in TBS and incubated with the coverslips at 37° C. for 1 hr. By observing cells with a confocal microscope, the images of cell morphology were obtained by detection of actin and the images of nuclei of the cells and the infected bacteria were obtained by detection of DNA.

### Measurement of Phosphorylation.

Part of the cultured cells were subjected to Western blotting in the same manner as in Example 1 and activation of PI3 kinase was examined using the degree of Akt phosphorylation as the indicator.

### Measurement of Cytokine Expression.

Total RNA was isolated from the cultured cells using ISOGEN RNA extraction reagent (Nippon Gene) and cDNA was synthesized using ReverTra Ace cDNA synthesis kit (Toyobo), with 5 μg of total RNA as a template. The expression level of mRNA for IL-12 (IL-12p40) was relatively quantified by performing real-time PCR using the Light Cycler 2.0 PCR system (Rosch) and the following primer pairs, with the aforementioned synthesized cDNA as another template:
p40 forward primer: CAGAAGCTAACCATCTCCTGGTTTG (SEQ ID NO: 6);
p40 reverse primer: CCGGAGTAATTTGGTGCTCCACAC (SEQ ID NO: 7);
Reaction condition: an initial denaturation at 95° C. for 0.5 min, followed by 45 cycles of 10 sec at 95° C., 30 sec at 57° C., and 30 sec at 72° C.
   Co-Culture with espH Mutant and Measurement.

Using the espH mutant of EPEC described in Example 1, a co-culture experiment was performed by using BMDC in the same manner as above. The resulting infected cells were subjected to immunofluorescence staining and their phosphorylation was measured in respective manners as described above.

### Results.

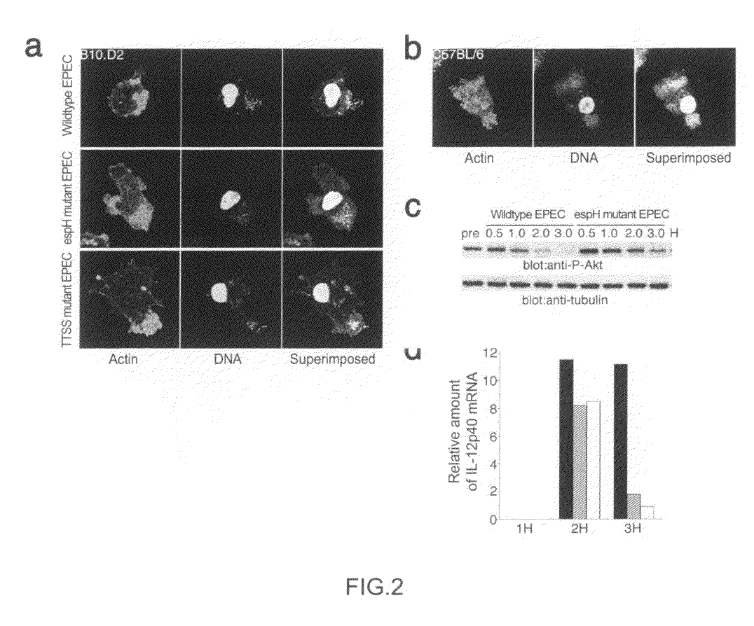

In the primary cultured BMDCs isolated from the bone marrow, the degree of suppression of PI3 kinase activity observed when infected with the wild-type EPEC was decreased in the case infected with the espH mutant (FIG. 2c). Further, the increase in IL-12 expression due to the bacterial infection was sustained beyond 2 hr after infection with the wild type, but it was decreased within 3 hr in the case with the TTSS mutant, as well as the espH mutant (FIG. 2d). These results showed that, like in Example 1, by introducing the EspH protein into dendritic cells, PI3 kinase activity can be inhibited within dendritic cells and IL-12 synthesis can be promoted.

In addition, as a result of observation of infection with EPEC or its mutant, it was shown that the wild-type bacteria adhered to the surface of the cells to form small colonies (FIG. 2a top and b), whereas the TTSS mutant strain was phagocytosed by the cells (FIG. 2a bottom), and when the espH mutant was co-cultured, it formed small colonies without being phagocytosed, like the wild-type (FIG. 2a middle). These findings indicated that the espH mutant can be infected with cells like the wild-type, and, therefore, that the phenotype of an espH mutant is not caused by the inability of EPEC to infect cells.

### Example 3

### Experiment of Infection of Mice with Citrobacter rodentium

In this Example, it has been shown by examining the immune responses in mice that cell-mediated immune responses in a mouse can be increased by administration of the EspH protein to the mouse by means of infecting Citrobacter rodentium into individual mice.

### Oral Infection and Measurement of Infection in the Colon.

BALB/c mice and B10.D2 mice were obtained from CLEA Japan and Japan SLC, respectively. All the mice were housed for a week, in accordance with the guidelines established by the University of Tokyo, in the Laboratory Animal Research Center, the Institute of Medical Science, the University of Tokyo. Citrobacter rodentium EX-33 strain (hereinafter abbreviated as C. rodentium; provided by Sasakawa Lab. Institute of Medical Science, University of Tokyo) was cultured at 37° C. overnight. 200 μl of the culture (a volume equivalent to 2-3×10⁸ colony-forming unit (cfu) per mouse) was infected into the mice by oral administration through the diet. Mice were sacrificed after continued housing for a designated period of time. A segment of the distal colon was excised at 4.5 cm from the rectum and the fecal pellets were removed by washing with PBS. After weight measurement and macroscopic observation of the tissue, homogenization was performed with a Potter Elvehjem homogenizer (digital homogenizer, As One). The homogenate was serially diluted with cold PBS and then seeded on MacConkey's agar plates (Difco Laboratories). The number of the colonies produced on the plate was counted to determine CFU per mouse to be used as an indicator of the degree of infection. Meanwhile, the tissue excised similarly 12 days after infection was fixed with 10% neutral buffered formalin, sectioned, subjected to hematoxylin-eosin tissue staining, and microscopically observed.

### Quantification of IFN-γ.

The mesenteric lymph node (MLN) cells were isolated from the mesentery of the mice on day 12 of infection using a syringe needle. After cells were gently suspended, tissue fragments were removed by passing through a 100-μm mesh. Cells were then transferred to RPMI 1640 medium and culture was started at 37° C. Subsequently, the MLN cells were stimulated with bacterial lysate at 37° C. for 72 hr. The concentration of mouse TNF-γ in the culture medium was determined using the Quantikine M ELISA kit (R&D Systems).

Construction of an espH Mutant of C. rodentium.

An espH mutant of C. rodentium was constructed in the following method. For the mutant, while either a hypomorph or a amorph may be used, an amorph with a defect in the espy gene was constructed in this case.

First, the DNA fragment (espH-5) corresponding to the upstream region of the espH gene was amplified by performing PCR under the following reaction condition using the following primer pairs, with the chromosomal DNA of C. rodentium as template:
espH-5 forward primer: AACTGCAGAAGAGGAGCACTCGT (SEQ ID NO: 2);
espH-5 reverse primer: GCGTCGACCATGATACATCTCCC (SEQ ID NO: 3);
Reaction condition: an initial denaturation at 94° C. for 2 min, followed by 30 cycles of 60 sec at 94° C., 60 sec at 58° C., and 120 sec at 72° C.

Similarly, the DNA fragment corresponding to the downstream region of the espH gene (espH-3) was amplified by PCR using the following primer pairs and reaction condition:
espH-3 forward primer: GCGTCGACCCTTTGTCAGGCATG (SEQ ID NO: 4);
espH-3 reverse primer: GCTCTAGAAATCTGCTCCTGCCG (SEQ ID NO: 5);
Reaction condition: an initial denaturation at 94° C. for 2 min, followed by 30 cycles of 60 sec at 94° C., 60 sec at 58° C., and 120 sec at 72° C.

The DNA fragment between the restriction enzyme PstI cleavage site and the SalI cleavage site from the obtained espH-5, as well as the DNA fragment between the SalI cleavage site and the XbaI cleavage site from the obtained espH-3, were each excised by digestion with the corresponding restriction enzymes. These two DNA fragments were ligated at the XbaI sites and the ligated DNA fragments were inserted into a cloning site of a sucrose-sensitive suicide vector pCACTUS (provided by Sasakawa Lab., Institute of Medical Science, University of Tokyo). The inserted espH-derived DNA fragment contains a deletion at amino acid positions 10 through 171 of the EspH protein encoded by the espH gene.

The recombinant expression vector pCACTUS-espH thus obtained was introduced into C. rodentium by electroporation. The resulting transformant was cultured overnight on LB plates in the presence of 5 μg/ml trimethoprim to select bacteria into which pCACTUS-espH had been introduced. It is known that the pCACTUS vector, having a temperature-sensitive replication site, cannot replicate at 30° C. or higher and resultantly is incorporated into the host E. coli genome. By utilizing this mechanism, introduction of pCACTUS-espH into the genome was induced by culturing the selected bacteria overnight at 42° C. The whole pCACTUS-espH was thus incorporated into the espH gene site of C. rodentium. Next, by taking advantage of sucrose sensitivity of pCACTUS, the pCACTUS-espH which had been incorporated into the genome was again removed from the genome. During this procedure, the genomes in which the pCACTUS-espH had been incorporated usually return to the original state, but there exist some genomes into which the mutated espH gene remains stably at a certain probability. Thus, bacteria in which the espH mutant gene had been stably incorporated into their genome were identified as follows: a PCR was performed by using the genome finally obtained from the colonies as a template together with the espH-5 forward primer and espH-3 reverse primer in a reaction condition of initial denaturation at 94° C. for 2 min, followed by 30 cycles of 60 sec at 94° C., 60 sec at 58° C., and 120 sec at 72° C.; the amplified DNA fragments were then electrophoresed; and comparison was made between the lengths of respective PCR products. A plurality of the bacteria thus obtained were further cultured, and the mutation of the gene was confirmed using the PCR method again to obtain an espH mutant of C. rodentium. This mutant exhibited a phenotype of a defect in the function of the EspH protein.

Infection with espH Mutant and its Measurement.

Using the C. rodentium espH mutant thus obtained, an infection experiment was performed in BALB/c mice in the same manner as above. In the resulting infected mice, measurement of bacterial infection, tissue observation, and cytokine quantification in the colon were performed by respective methods as described above.

### Results.

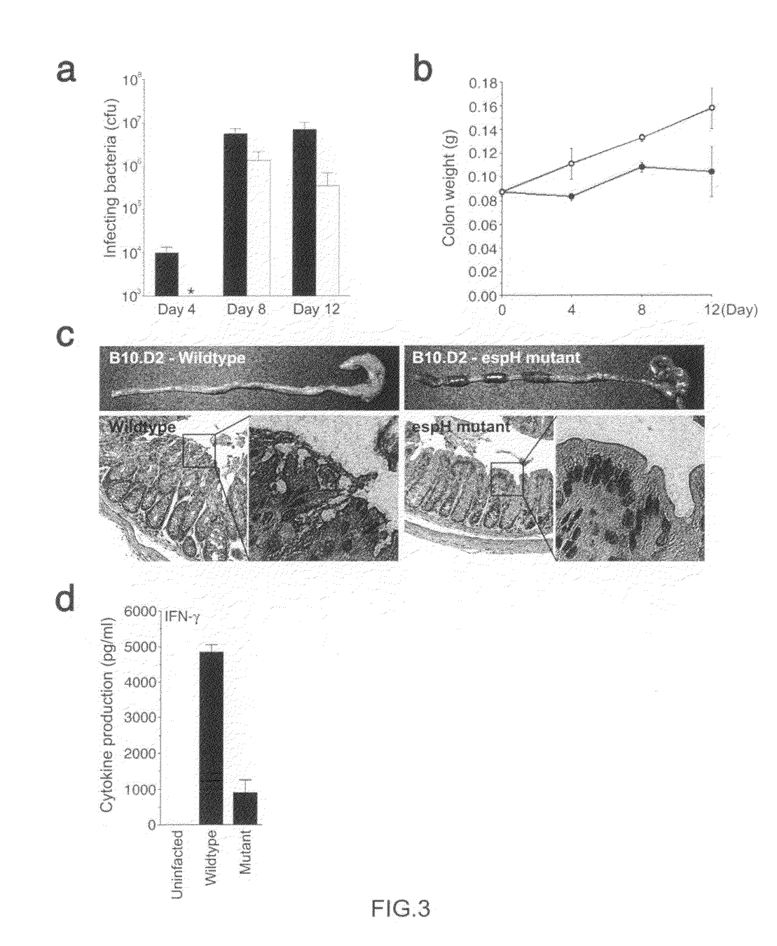

Wild-type C. rodentium, when infected into the mouse colon, rapidly grew within eight days (FIG. 3a, black vertical bars) and the weight of the tissue was increased (FIG. 3b, white circles). In contrast, when the espH mutant was infected, the number of bacteria in the colon was smaller and the degree of the increase in the tissue weight was also smaller than the wild-type (FIG. 3a, white vertical bars; and FIG. 3b, black circles). In the mice infected with the wild-type, a macroscopic observation showed that solid feces in the colon had disappeared and the whole colon was edematous; and a histopathological observation showed that the infection site had pathological lesions (FIG. 3c left). In contrast, in the mice infected with the espH mutant, the healthy colon containing solid feces was observed, and no pathological lesions was found in the tissue image (FIG. 3c right). These results suggest that the espH protein promotes proliferation of bacteria, causing the pathological lesions at the infection site.

Further, as a results of cytokine quantification in the MLN, IFN-γ, which had not been detected before infection, was markedly expressed after the infection with the wildtype, but almost no is expression occurred with the mutant (FIG. 3d). Since IFN-γ is induced to be expressed by IL-12 in Th1 cells having the function of enhancing cell-mediated immune responses and plays the pivotal role in Th1 cell differentiation and proliferation, it was indicated that the EspH protein enhances cell-mediated immune responses in mice.

In summary, it was clarified that the EspH protein secreted by the C. rodentium infected into the mouse colon increases cell-mediated immune responses in a mouse and, therefore, that the EspH protein is effective as an activator of cell-mediated immune responses.

### Example 4

### Infection Experiment of PI3 Kinase Knockout Mice with C. rodentium

In this Example, PI3 kinase knockout mice having the genetic background of BALB/c mice, which are known to be unaffected by infection with C. rodentium because of the predominance of humoral immune responses over cell-mediated immune responses, are infected with C. rodentium and its effect is examined. Through this experiment, the followings are demonstrated: (a) the predominance of humoral immune responses in BALB/c mice is associated with PI3 kinase and thus, by inhibiting PI3 kinase, humoral immune responses can be suppressed; and (b) since the function of the EspH protein is exhibited via PI3 kinase in mice as well, PI3 kinase is inhibited by administration of the EspH protein, causing a resultant downstream event.

### Construction of Knockout Mice.

Mice lacking the p85α regulatory subunit of PI3 kinase were constructed in the following manner (For details, see Terauchi, Y., Tsuji, Y., Satoh, S. et al. (1999) “Increased insulin sensitivity and hypoglycemia in mice lacking the p85a subunit of phosphoinositide 3-kinase.” Nature Genetics 21: 230-235). The Pik3r1 gene encoding p85α was isolated from the mouse D3 genomic library. A cassette containing a neomycin resistance gene was inserted between restriction enzyme PstI cleavage sites flanking exon 1A of the Pik3r1 gene. The gene lacking p85α thus constructed was introduced into embryonic stem cells by homologous recombination. Using the resulting p85α (+/−) embryonic stem cells, PI3 kinase knockout mice were constructed. The resulting knockout mice were backcrossed to the BALB/c background for 12 generations, whereby the genetic background of the knockout mice was substituted with the background of BALB/c mice. The mouse individual thus constructed which is homozygous for the mutated p85α gene exhibits a PI3 kinase-deficient phenotype, and is herein referred to as a p85α knockout mouse. A mouse individual which is heterozygous for the mutated p85α gene shows a normal PI3 kinase function in its phenotype, like a wild-type mouse, and herein referred to as a p85α heterozygote.

### Infection Experiment.

An infection experiment was performed in the same manner as in Example 3 by infecting wild-type BALB/c mice and p85α knockout mice with wild-type C. rodentium or its espH gene-deficient mutant. For each of the infected mice, counting of the number of infected bacteria, measurement of the tissue weight of the excised colon, and macroscopic and histological observations of them were performed in the same manner as in Example 3.

### Results.

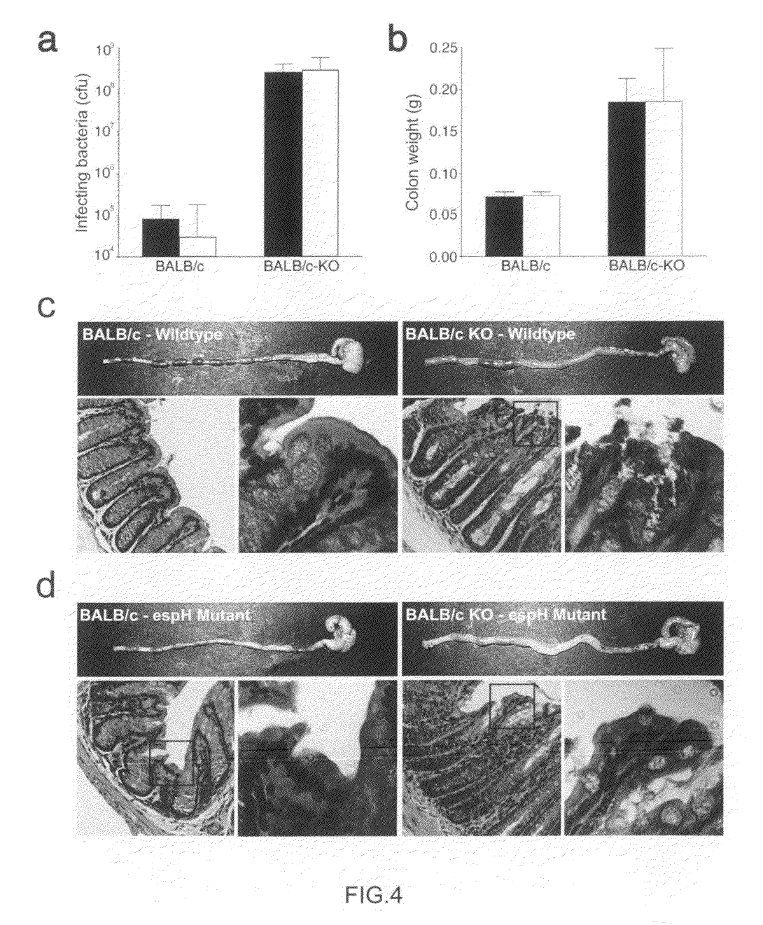

p85α knockout mice on a BALB/c genetic background were infected with wild-type C. rodentium. The knockout mice exhibited a high degree of infection comparable to that of wild-type B10.D2 mice (FIG. 4). This result indicates that the PI3 kinase is involved in the predominance of humoral immune response in BALB/c mice, and thus by inhibiting PI3 kinase with the espH protein, humoral immune responses can be suppressed.

Meanwhile, when the knockout mice were infected with the C. rodentium espH mutant, the mice did not show a decrease in infectivity due to mutation of the espH gene, which was observed in Example 3 (FIG. 4). Further, also as a result of observation of the excised colon, the knockout mice (FIG. 4c right) exhibited a higher infection than the wild-type mice (FIG. 4c left) regardless of the presence or absence of the espH mutation. These findings confirmed that also in a mouse individual, the target of the EspH protein is the PI3 kinase.

### Example 5

### Infection Experiment of Bone Marrow-Transplanted Chimeric Mice with C. rodentium

By performing an experiment of infection of chimeric mice generated by transplanting the bone marrow of p85α knockout mice to wild-type mice, it has been shown that the target of the EspH protein possessed by C. rodentium is bone marrow-derived cells, especially dendritic cells.

### Transplantation of the Bone Marrow.

Cells were harvested by the method described in Example 2 from the bone marrow of the femur of either p85α knockout mice or p85α heterozygous mice with BALB/c genetic background which had been generated by the method described in Example 2. After red blood cells were removed by lysis with ammonium chloride buffer, 10⁷ bone marrow cells from each were suspended in 0.15 ml each of PBS. Meanwhile, another group of p85α heterozygous mice were exposed to 4.5 Gy X-ray irradiation with an X-ray irradiation system (Hitachi Medical Corp.) and then received an intravenous injection of either of the aforementioned bone marrow cell suspensions.

### Infection Experiment.

Using the two types of bone marrow-transplanted chimeric mice thus obtained, an infection experiment was performed with C. rodentium in the same manner as in Example 3. In the respective infected mice, measurement of bacterial infection and tissue observation were performed in the method as described in Example 3.

### Results.

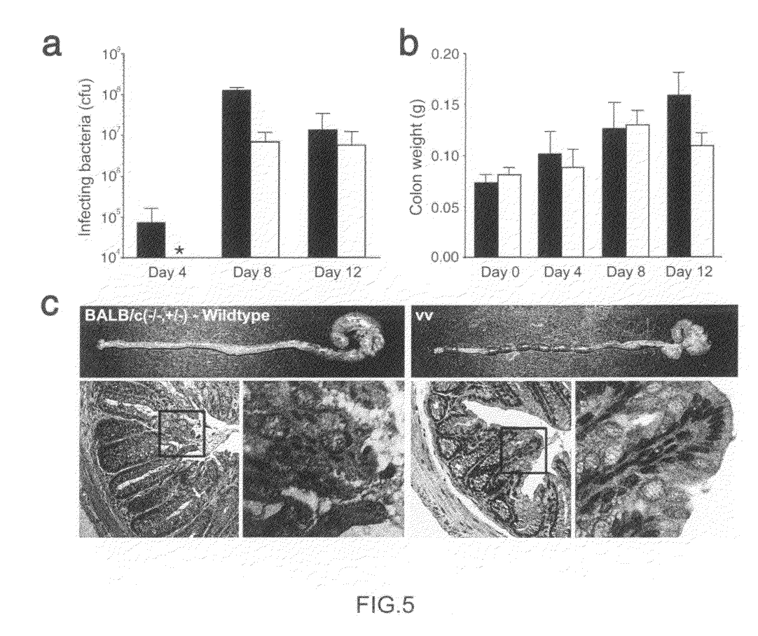

Since all bone marrow cells had been once killed by a lethal dose of UV irradiation, almost all the cells in the bone marrows, including dendritic cells, of the chimeric mice generated by transplanting the bone marrow of knockout mice to p85α heterozygous mice, are those derived from the knockout mice. An experiment of infection of such chimeric mice exhibited a higher infectivity in the chimeric mice, like knockout mice per se, than wild-type mice (FIG. 5a, b) and developed pathological lesions associated with infection in the colon, which would not develop in wild-type mice (FIG. 5c). These findings confirm that the target of the EspH protein possessed by C. rodentium is bone marrow-cell derived cells, which include at least dendritic cells, as revealed by Examples 1, 2 etc. Therefore, it was shown that dendritic cells are suitable as the target cells for the expression of the EspH protein.

### Example 6

### Induction Experiment of Differentiation of T Cells Derived from Knockout Mice

By inducing differentiation of T cells isolated from individual mice, it has been demonstrated that expression of macrophage inflammatory protein-2 (MIP-2), a chemokine secreted from T cells, is regulated by PI3 kinase in T cells.

### Isolation and Stimulation of Mouse Spleen Cells.

Spleen cells were isolated from each of p85α heterozygous mice and p85α knockout mice generated by the method as described in Example 4, and red blood cells were removed by lysis with ammonium chloride buffer. Using anti-CD4 antibody microbeads (Daiich Pure Chemicals), CD4+ T cells were isolated with an AutoMACS Cell Sorter (Miltenyi Biotec) and cultured in RPMI1640 medium containing 10% fetal bovine serum. These cultured cells were stimulated by simultaneous addition of either anti-CD3 antibody and CD28 antibody (10 mug/ml each; BD PharMingen) or Phorbol myristate acetate (PMA, 50 ng/ml, Calbiochem) and Ionomycin (1 μg/ml, Calbiochem). Forty-eight hours after start of the stimulation, culture supernatants were recovered and the concentration of MIP-2 contained in the supernatants was measured by ELISA using anti-MIP-2 antibody (R&D Systems).

### Results.

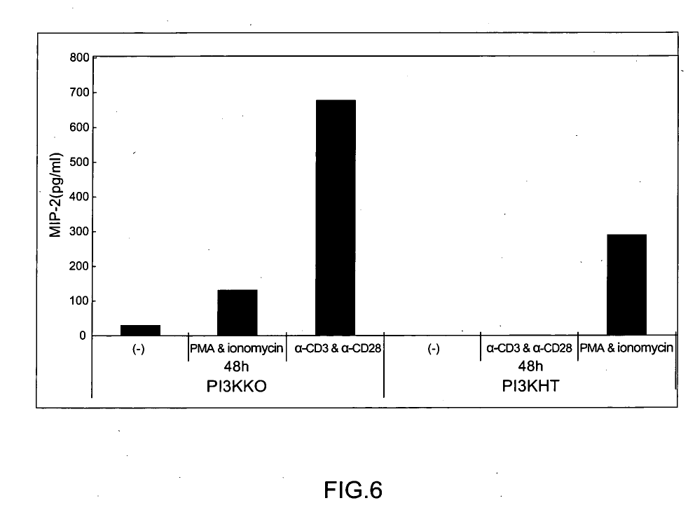

In the cultured T cells derived from knockout mice lacking PI3 kinase, MIP-2 expression was increased by the addition of the differentiation inducers, and was more markedly increased by the stimulation with the antibodies (FIG. 6 left). In contrast, in the experiment using T cells derived from wild-type mice, addition of the differentiation inducers caused no increase in MIP-2 expression and the stimulation with the antibodies resulted in a smaller degree of increase than that when knockout mice-derived T cells were used (FIG. 6 right).

These results indicated that PI3 kinase in T cells has the effect of suppressing chemokine expression, and, therefore, that T cells are suitable as a target for induction of chemokine secretion by inhibiting its PI3 kinase activity.

It should be noted that, the method for increasing chemokine expression in T cells is not limited as long as it can inhibit PI3 kinase activity within the T cells. Expressing the EspH protein serves to exemplify the embodiment.

### Example 7

### Suppression of Cytokine Expression in BMDCs by GSK3β Inhibitor

In this Example, it has been demonstrated that, by inhibiting GSK3β in isolated dendritic cells, expression of PI3 kinase-dependent inflammatory cytokines is suppressed.

### Culture Experiment.

Bone marrow-derived dendritic cells (BMDCs) were isolated from wild-type mice by the method as described in Example 2. Next, the isolated BMDCs were infected with wild-type EPEC or its espH mutant by co-culture in the same manner as in Example 1. The phosphorylation level of GSK3β in total lysates from each of the infected cells was determined by Western blotting using anti-phosphorylation Ser9-GSK3β antibody (Cell Signaling). Meanwhile, after pretreatment for 1 hr with 10 nM GSK3β inhibitor SB216763 (Sigma) or 5 mM LiCl, the isolated BMDCs were stimulated with a 1 μg/ml LPS solution for 24 hr. The content of IL-12 in the culture supernatants after the stimulation was quantified using the ELISA kit specific to anti-mouse IL-12p70.

### Results.

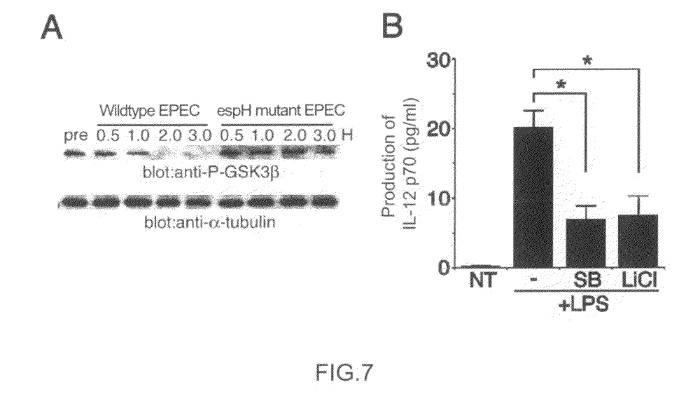

As shown in FIG. 7A, in BMDCs infected with wild-type EPEC, dephosphorylation of phosphorylated GSK3β rapidly progressed (upper row left), whereas in BMDCs infected with the espH mutant dephosphorylation did not take place (upper row left). Further, as shown in FIG. 7B, in BMDCs activated by LPS stimulation, inhibition of GSK3β activity by the addition of SB216763 or LiCl resulted in a decrease in the production of IL-12.

It was therefore clarified that, in dendritic cells, by inhibiting GSK3β, expression of inflammatory cytokines can be suppressed.

### Example 8

### Suppression of Infection of Mice with C. rodentium by Li Ions

In this example, it has been demonstrated that by administering Li ions to mice infected with pathogenic E. coli, production of inflammatory cytokines is suppressed and thereby symptoms associated with the infection are relieved.

### Bacterial Infection Experiment.

Wild-type B10.D2 mice were orally infected with C. rodentium by the method as described in Example 3. Three days after infection, the mice were divided into three groups, and one group was sacrificed and the weight of the colon cleared of lumps of feces was measured. Then, for one group of the two remaining groups, serving as the experimental group, drinking water was replaced by 30 mM LiCO₃-containing water, while the other group, serving as the control group, received reprocessed water. Each group was housed for three more days, and six days after the infection, the mice in the experimental and control groups were sacrificed and the weight of the colon was measured. A part of each of the colon tissues was fixed, frozen, sectioned, subjected to hematoxylin-eosin tissue staining, and microscopically observed, by the method as described in Example 3.

### Measurement of Infectivity and Cytokines.

The remaining tissue sample was plated on MacConkey's agar after homogenization by the method as described in Example 3. The number of bacteria adhered to the mouse colon was determined by counting the number of colonies formed. Meanwhile, MLN cells were isolated by the method as described in Example 3 from the mice 6 days after the oral infection. The isolated MLN cells were stimulated in vitro with C. rodentium lysate and IFN-γ produced was quantified by ELISA.

### Results.

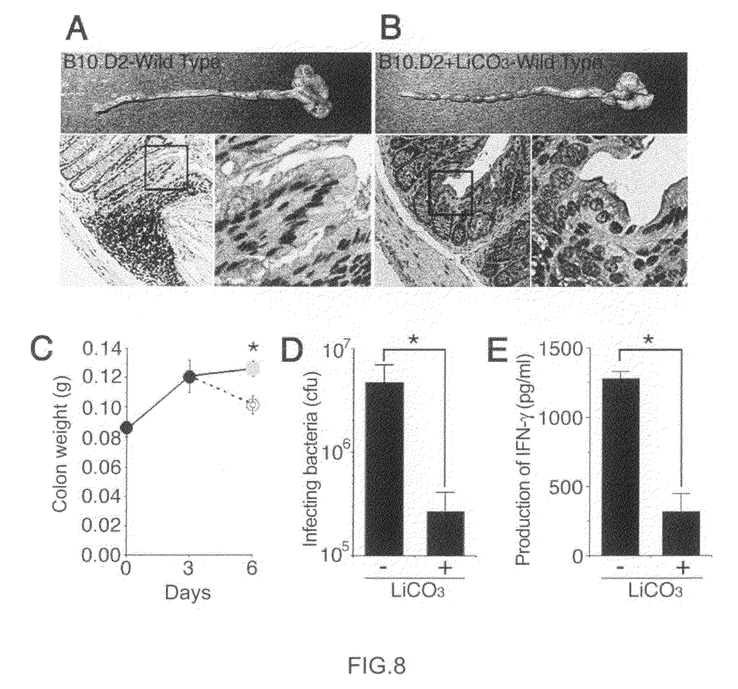

FIGS. 8A and B show images obtained by microscopic observation of the colons excised from the mice in the experimental and control groups on day 6 after the infection. FIG. 8C is a line graph showing the weight of the colon on days 0, 3, and 6 after infection. These results indicate that, in the mice in the experimental group receiving LiCO₃, the symptom of bacterial infection in the colon was relieved as compared with the mice in the control group not receiving LiCO₃. Also in the mice in the experimental group receiving LiCO₃, the number of infecting bacteria in the colon was significantly decreased compared with the control group, as shown in the bar graph of FIG. 8D. Further, in the mice in the experimental group, the expression level of IFN-γ in the lymph node cells was decreased compared with mice in the control group, as shown in the bar graph of FIG. 8E. Since IFN-γ is induced to be expressed by IL-12, an inflammatory cytokine in Th1 cells which have the function of enhancing cell-mediated immune responses, the results of this experiment revealed that, not only in the cultured cells of Example 7 but also in vivo, IL-12 expression is suppressed by administration of Li ions. Further, since IFN-γ plays the pivotal role in Th1 cell differentiation and proliferation, Li ions were shown to suppress the inflammatory cytokine production as well as the cell-mediated immune response, thereby activating the humoral immune responses, in mice.

In conclusion, Li ions have the effect of regulating immune responses, and thus is useful as a suppressor of cell-mediated immune responses, an activator of humoral immune responses, and a suppressor for suppressing proliferation of pathogenic bacteria.

1-22. (canceled)

23. A method for inhibiting in vivo synthesis of a PI3 kinase-dependent inflammatory cytokine in a vertebrate, comprising a step of administering Li ion to the vertebrate.

24. The method of claim 23, wherein the cytokine is IL-12.

25. The method of claim 23, wherein the Li ion is administered by injection or oral administration.

26. A method for suppressing cell-mediated immune responses in a vertebrate, comprising a step of administering Li ion to the vertebrate.

27. The method of claim 26, wherein the Li ion is administered by injection or oral administration.

28. A method for activating humoral immune responses in a vertebrate, comprising a step of administering Li ion to the vertebrate.

29. The method of claim 28, wherein the Li ion is administered by injection or oral administration.

30. A therapeutic method for treating a disease resulting from synthesis of a PI3 kinase-dependent inflammatory cytokine, comprising a step of administering Li ion to the vertebrate.

31. The therapeutic method of claim 30, wherein the cytokine is IL-12.

32. The therapeutic method of claim 31, wherein the Li ion is administered by injection or oral administration.

33. A therapeutic method for a disease resulting from an increase in the ratio of a cell-mediated immune response to a humoral immune response, comprising a step of administering Li ion to the vertebrate.

34. The therapeutic method of claim 33, wherein the disease is a disorder resulting from proliferation of pathogenic E. coli, an immune-mediated inflammatory disorder (IMID), a habitual miscarriage, an organ-specific autoimmune disease based on delayed type hypersensitivity, a hepatic disorder, or arteriosclerosis.

35. The therapeutic method of claim 33, wherein the Li ion is administered by injection or oral administration.

36. A method for suppressing in vivo proliferation of pathogenic E. coli in a vertebrate, comprising a step of administering Li ion to the vertebrate.

37. The method of claim 36, wherein the Li ion is administered by injection or oral administration.
